(19) United States
(12) Patent Application Publication
(10) Pub. No.: US 2010/0228320 A1
(43) Pub. Date: Sep. 9, 2010

(54) Metallic Implant with Reduced Secondary Electron Generation

(75) Inventor: Martin Zimmerling, Patsch (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/710,006

(22) Filed: Feb. 22, 2010

Related U.S. Application Data
(60) Provisional application No. 61/157,713, filed on Mar. 5, 2009.

(51) Int. Cl. A61N 1/16

(52) U.S. Cl. 607/55

(57) ABSTRACT

An implant includes a housing and at least one electronic component. The housing is made of a first material and has an outer surface and an inner surface. The shield is positioned within the housing proximate the inner surface, the shield made of a second material. The second material has an atomic number greater than the atomic number of the first material.

### CROSS-REFERENCE TO RELATED APPLICATIONS

This application claims priority to U.S. Provisional Patent Application No. 61/157,713 filed Mar. 5, 2009, the disclosure of which is incorporated by reference herein in its entirety.

### FIELD OF THE INVENTION

The present invention relates to protecting implants from irradiation, and more particularly, to an implant that includes proper shielding with regard to secondary electron generation and braking radiation (also referred to retardation radiation or bremsstrahlung), such that adjacent tissue and electronic components within the implant are protected.

### BACKGROUND ART

A patient having an electronic implant may require therapeutic irradiation, such as when undergoing cancer treatment. However, such treatment is problematic in that damage may occur to surrounding tissue or the implant's electronic components.

FIG. 1 shows a cross-section of an exemplary electronic implant 101 inserted underneath the skin 107 (prior art). The implant 101 includes a hermetic, metallic housing 103. The metallic housing 103 provides a certain level of protection for the implant from ionizing radiation, such as when undergoing gamma radiation therapy to treat cancer. However, as a byproduct, secondary electrons are produced in a phenomenon referred to as secondary emission. This secondary electron generation can damage surrounding tissue. (Biocompatible) materials having a low atomic number, such as titanium and niobium, generate fewer secondary electrons than materials with high atomic numbers when exposed to radiation, and are thus preferred as a housing material in this regard.

Within the implant housing 103 are one or more electronic components 105, such as various semiconductors, sensors, power supplies (such as batteries which may be rechargeable), and/or microphones. There is a risk that any secondary electrons generated by interaction of the gamma rays with the housing 103 may damage the electronic components 105. Furthermore, when exposed to radiation, the housing 103 may produce highly penetrating braking radiation, further damaging the electronic components 105.

To avoid damage to surrounding tissue and the internal electronics 105, it is standard clinical procedure to externally shield the implant 101, and to position the radiating beam so as to minimize exposure to the implant 101. However, this is not always an option, particularly when irradiation is required in close proximity to the implant 101. Implants having non-metallic housings have also been manufactured, however these implants provide little protection for those electronic components susceptible to ionizing irradiation. In some cases semiconductor technology robust against ionizing irradiation may also be used, but at increased cost. On the other hand, semiconductor technology optimized for low power or high speed applications may be more susceptible against ionizing irradiation.

### SUMMARY OF THE INVENTION

In accordance with one embodiment of the invention, an implant includes a housing having an outer surface and an inner surface. The housing is made of a first material. A shield is positioned within the housing proximate the inner surface, the shield made of a second material. The second material has an atomic number greater than the atomic number of the first material. An electronic component is located within the housing.

In accordance with related embodiments of the invention, the housing may be hermetic. The first material and the second material may be a metal or a metal alloy. The first material may be a biocompatible metal like titanium or niobium. The second material may be tantalum, iridium, platinum or tungsten. The atomic number of the first material may be less than 47. The atomic number of the second material may be at least equal to 47. Additional layers of materials of alternating low and high atomic numbers may be provided as additional shielding. The shield may not mask the entire housing. Thickness of the shielding (with second material) may be designed either to achieve highest possible protection of electronic components inside the implant, or it may be optimized to minimize back-scattering of secondary electrons through the first layer towards the surrounding tissue, or to achieve a good tradeoff between these two requirements. The electronic component may be a semiconductor, battery, sensor, and/or microphone. The implant may be a cochlear implant.

In accordance with another embodiment of the invention, an implant includes a housing having an outer surface and an inner surface, the housing made of a first material. A shield is positioned proximate the outer surface of the housing, the shield made of a second material. The second material has an atomic number less than the atomic number of the first material. An electronic component is positioned within the housing.

In accordance with related embodiments of the invention, the housing may be hermetic. The first material and the second material may be a metal and/or a metal alloy. The first material may be iridium, platinum, tantalum or tungsten. The second material may be titanium or niobium. The atomic number of the first material may be at least equal to 47, with the atomic number of the second material being be less than 47. The shield may not cover the entire housing. The electronic component may include a semiconductor, battery, sensor and/or microphone. The implant may be a cochlear implant.

In accordance with yet other embodiments of the invention, an implant includes a housing. The housing is made of a metallic material with a window made of a non-metallic material. An electronic circuit is positioned within the housing, under the non-metallic portion of the housing. The non-metallic material may be, for example, a ceramic.

### BRIEF DESCRIPTION OF THE DRAWINGS

The foregoing features of the invention will be more readily understood by reference to the following detailed description, taken with reference to the accompanying drawings, in which:

FIG. 1 shows a cross-section of an implant with a hermetic metallic housing (prior art);

FIG. 2 shows a cross-section of an implant that includes a metallic shield made of a material with a high atomic number, in accordance with an embodiment of the invention;

FIG. 3 shows a cross-section of an implant that includes a metallic housing with a non-metallic window, in accordance with an embodiment of the invention; and

FIG. 4 shows a cross-section of an implant that includes a shield made of a material with low atomic number, in accordance with an embodiment of the invention.

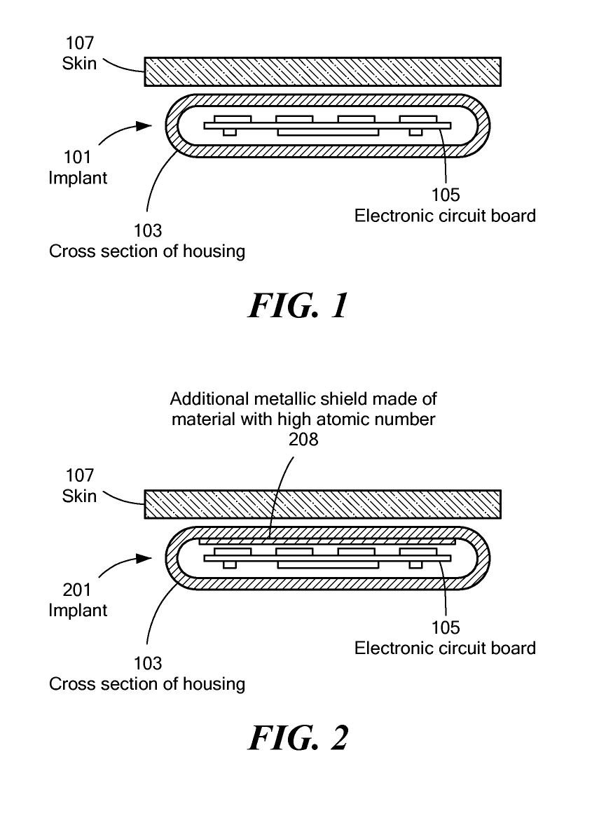

### DETAILED DESCRIPTION OF SPECIFIC EMBODIMENTS

In illustrative embodiments, an implant includes enhanced shielding to minimize damage to surrounding tissue and/or implant electronics caused by secondary electron generation. Details are discussed below.

FIG. 2 shows a cross-section of an implant 201 located under the skin 107 that includes enhanced shielding from irradiation. The implant may be one of a variety of implants, including any kind of neuro-stimulator, spinal cord stimulator, deep brain stimulator and laryngeal pace maker. Further examples of implants include, without limitation, cochlear implant, a defibrillator, a cardioverter, a pacemaker, and a retinal implant.

The implant 101 includes a hermetic housing 203 and one or more electronic components 105. The electronic components 105 may include, for example, an electronic circuit board having one or more semiconductors, a microphone, a sensor, and/or a power supply, such as a battery which may be rechargeable. These electronic components 105 are often sensitive to radiation fields. For example, transistors are susceptible to malfunction because of defect trapping of charge carriers. Ferroelectrics may fail because of induced isotropy. Quartz oscillators may change frequency and magnetic materials may deteriorate because of hardening. Plastics used for electrical insulation may deteriorate.

The housing 203 is made of a first metallic material, such as a metal or metal alloy. The thickness of the first material is such that it absorbs (at least part of the) gamma rays, thereby serving to at least partially protect the electronic components 105. Optimally, the thickness of the first material slightly exceeds the penetration depth of the gamma rays. However, in reality, there is a reasonable upper limit for the thickness of an implant, so that there is a compromise between the gamma ray shielding functionality and the thickness of the implant.

The first material advantageously may have a low atomic number to minimize secondary electron generation which may harm adjacent tissue. For example, the atomic number of the first material may be, without limitation, less than 47. Materials having low atomic number that may be used for the housing 203 include, for example, titanium and niobium.

In illustrative embodiments of the invention, the implant 201 includes an additional metallic shield 208 positioned within the housing 203. The shield 208 may be positioned, for example, proximate to/underlying the inner surface of the housing 203 of the implant 201. In various embodiments, the shield 208 may be positioned substantially adjacent the inner surface of the housing 203, and, without limitation, may contact the inner surface of the housing 203. In various embodiments, the shield 208 may be distanced from the inner surface of the housing 203.

The shield 208 functions to absorb braking radiation and secondary electrons generated by the first material. The shield 208 is preferably a layer of a second material that has an atomic number greater than the atomic number of the first material. The higher the atomic number, the better the absorption of braking radiation. In various embodiments, the atomic number of the second material may be at least equal to 47. The second material may be, without limitation, a metal or a metal alloy, such as platinum, iridum, gold, tantalum or tungsten.

The shield 208 on the inner side of the housing may not cover the complete implant housing 203. Instead, the shield 208 may only be present in those areas that protect radiation-sensitive electronic components.

Additional layers of materials of alternating low and high atomic numbers may be provided as additional shielding. These additional layers of material may be situated outside and/or inside of housing 203. In preferred embodiments, the external outer layer of material of implant 201 is a material of low atomic number, such as lower than 47, such that fewer secondary electrons are produced that are harmful to adjacent tissue.

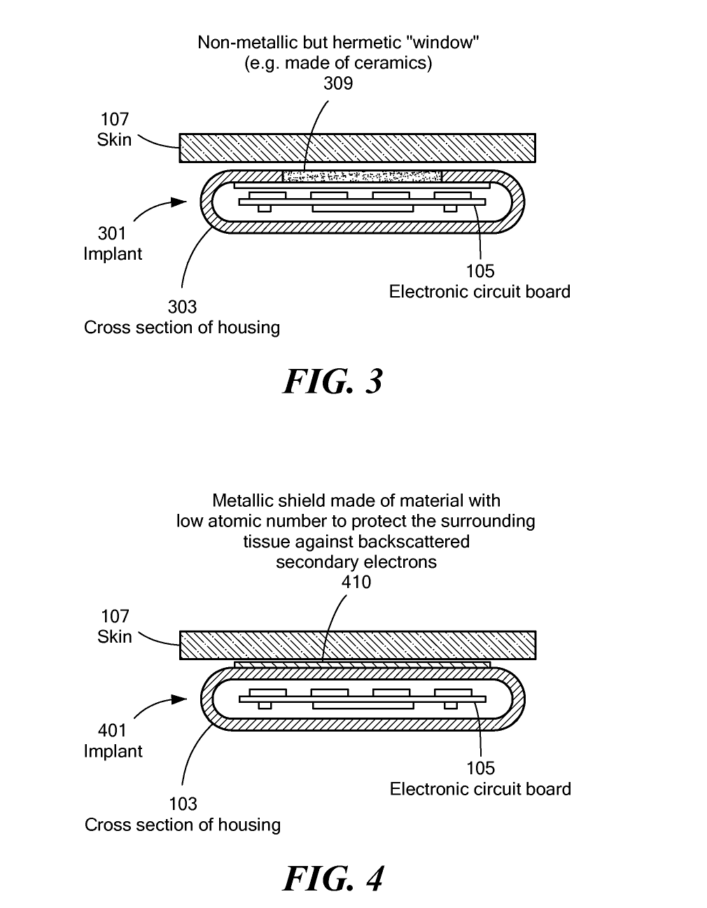

FIG. 3 shows a cross-section of an implant 301 that includes a metallic housing 303 with a non-metallic window 309, in accordance with another embodiment of the invention. Both the housing 303 and window 309 are hermetic, sealing and protecting electronic components 105 within the implant 301.

The non-metallic window 309 is positioned over the radiation-sensitive components to minimize the harmful effects of secondary electron generation and braking radiation. The window 309 itself may provide a certain level of protection against gamma or other radiation. However, when implementing such a window 309, there may be a tradeoff between the benefits of reduced secondary electron generation/braking radiation versus less protection against, for example, gamma radiation. The window 309 may include, without limitation, a ceramic or a ceramic compound. In alternative embodiments, the window may include some degree of metallic material.

FIG. 4 shows a cross-section of an implant 401 that includes a shield 410 proximate to, and overlaying, the outer surface of implant housing 103, in accordance with one embodiment of the invention. The shield 410 is made of a material having a low atomic number compared to housing 103 material. The shield 410 serves to protect the surrounding tissue against backscattered secondary electrons.

Similar to above embodiments, the shield 410 may not cover the complete implant housing 203. Instead, the shield 410 may only be present in those areas that protect radiation-sensitive electronic components. In various embodiments, the shield 410 may be positioned adjacent the outer surface of the housing 203, and, without limitation, may contact the outer surface of the housing 203. In various embodiments, the shield 410 may be distanced from the outer surface of the housing 203.

In accordance with another embodiment of the invention, an implant housing may be made of an alloy of metals that combines both metals with relatively low and relatively high atomic numbers. Such a housing material may advantageously minimize secondary electron generation and/or braking radiation. Illustratively, a housing may be made of an alloy that includes titanium, aliminium, vanadium (e.g. TiAl6V4), niobium (e.g. TiAl6Nb7) and zirkonium.

The described embodiments of the invention are intended to be merely exemplary and numerous variations and modifications will be apparent to those skilled in the art. All such variations and modifications are intended to be within the scope of the present invention as defined in the appended claims.

What is claimed is:

1. An implant comprising:
a housing having an outer surface and an inner surface, the housing made of a first material;
a shield positioned within the housing proximate the inner surface, the shield made of a second material, the second material having an atomic number greater than the atomic number of the first material; and
an electronic component within the housing.

2. The implant according to claim 1, wherein the housing is hermetic.

3. The implant according to claim 1, wherein the first material and the second material are one of a metal and a metal alloy.

4. The implant according to claim 1, wherein the first material is one of titanium and niobium.

5. The implant according to claim 1, wherein the second material is one of iridium, platinum, tantalum, and tungsten.

6. The implant according to claim 1, wherein the atomic number of the first material is less than 47.

7. The implant according to claim 1, wherein the atomic number of the second material is at least equal to 47.

8. The implant according to claim 1, further comprising additional layers of shielding materials of alternating low and high atomic numbers.

9. The implant according to claim 1, wherein the shield does not mask the entire housing.

10. The implant according to claim 1, wherein the electronic component includes at least one of a semiconductor, sensor, battery, or microphone.

11. The implant according to claim 1, wherein the implant is a cochlear implant.

12. An implant comprising:
a housing having an outer surface and an inner surface, the housing made of a first material;
a shield positioned proximate the outer surface of the housing, the shield made of a second material, the second material having an atomic number less than the atomic number of the first material; and
an electronic component within the housing.

13. The implant according to claim 12, wherein the housing is hermetic.

14. The implant according to claim 12, wherein the first material and the second material is one of a metal and a metal alloy.

15. The implant according to claim 12, wherein the second material is one of titanium and niobium.

16. The implant according to claim 12, wherein the first material is one of platinum, iridium, tantalum, and tungsten.

17. The implant according to claim 12, wherein the atomic number of the second material is less than 47.

18. The implant according to claim 12, wherein the atomic number of the first material is at least equal to 47.

19. The implant according to claim 12, wherein the shield does not cover the entire housing.

20. The implant according to claim 12, wherein the electronic component includes at least one of a semiconductor, sensor, battery, or microphone.

21. The implant according to claim 12, wherein the implant is a cochlear implant.

22. An implant comprising:
a housing, the housing made of a metallic material with a window made of a non-metallic material; and
an electronic circuit with the housing, the electronic circuit positioned under the non-metallic portion of the housing.

23. The implant according to claim 22, wherein the non-metallic material is a ceramic.
